(19) United States
(12) Patent Application Publication
(10) Pub. No.: US 2026/0263829 A1
(43) Pub. Date: Sep. 10, 2026

(54) A PHOTOTHERAPY DEVICE INCLUDING PROJECTIONS FOR IMPROVING LIGHT DELIVERY TO A 3D SKIN SURFACE

(71) Applicant: TRANSDERMAL CAP, INC., Gates Mills, OH (US)

(72) Inventors: Michael I. Rabin, Gates Mills, OH (US); Jacob M. Rabin

(73) Assignee: TRANSDERMAL CAP, INC., Gates Mills, OH (US)

(21) Appl. No.: 19/165,250

(22) PCT Filed: Mar. 22, 2024
(86) PCT No.: PCT/US2024/021096

§ 371 Date: Sep. 15, 2025

Related U.S. Application Data
(60) Provisional application No. 63/491,548, filed on Mar. 22, 2023.

(51) Int. Cl. A61N 5/06

(52) U.S. Cl. CPC A61N 5/0616; A61N 2005/0647; A61N 2005/0652

(57) ABSTRACT

A phototherapy device is providing for illuminating a surface of a 3D skin surface when positioned adjacent the illuminated surface of the 3D skin surface. The phototherapy device includes a number of projections positioned to compress against a user's scalp to illuminate a user's scalp while reducing the presence of air between the projections and the user's scalp, thereby increasing optical penetration to the user's scalp. By utilizing phototherapy device according to the present disclosure, the projections advantageously bypass the user's hair and pushing against (also referred to as compressing) the scalp to increase light penetration by mechanical optical skin clearing.

### RELATED APPLICATIONS

This application claims the benefit of U.S. 63/491,548 filed on Mar. 22, 2023. Which is herein incorporated by reference in its entirety.

### TECHNICAL FIELD

The present disclosure relates generally to phototherapy devices and more particularly to a phototherapy device that delivers optical energy to human body tissues and in particular to a phototherapy device configured to illuminate a three-dimensional (3D) skin surface.

### BACKGROUND

Light therapy has been shown as an effective treatment to stimulate hair growth and to treat certain skin disorders. For example, the publication Avci, Pinar, et al. “Low-level laser (light) therapy (LLLT) for treatment of hair loss.” Lasers in surgery and medicine 46.2 (2014): 144-151 identifies many applications of low-level light therapy (LLLT) including wound healing, fat metabolism, brain stimulation, psoriasis treatment and hair regrowth. There are devices currently available in the market for delivering low level light therapy in order to stimulate hair growth. The available devices employ either a single or segmented substrate including multiple light sources for delivering light having a known therapeutic effect.

### SUMMARY

The available low-level light therapy (LLLT) devices suffer from many problems, including rigid substrates, flexible substrates that do not conformally cover the scalp, inability to bypass hair and deliver light to the scalp of patients with thinning hair, restricted airflow to the light source arrays, high cost to manufacture, and discomfort to the patient. The present disclosure details a device for providing increased light penetration to the user's scalp using projections.

The present disclosure provides a phototherapy device including projections for improving light delivery to a 3D skin surface by applying pressure to the 3D skin surface to improve optical clearing and to improve index of refraction matching by reducing a presence of air between the light source and the 3D skin surface.

While a number of features are described herein with respect to embodiments of the invention; features described with respect to a given embodiment also may be employed in connection with other embodiments. The following description and the annexed drawings set forth certain illustrative embodiments of the invention. These embodiments are indicative, however, of but a few of the various ways in which the principles of the invention may be employed. Other objects, advantages, and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention in which similar reference numerals are used to indicate the same or similar parts in the various views.

FIG. 1 is side view of a phototherapy device including projections and illuminating a three-dimensional (3D) skin surface.

FIG. 2 is an interior view of the phototherapy device including an elastomeric cap.

FIG. 3 is a top view of a lightboard of the phototherapy device.

The present invention is described below in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

### DETAILED DESCRIPTION

The present disclosure provides a phototherapy device configured to illuminate a surface of a 3D skin surface when positioned adjacent the illuminated surface of the 3D skin surface. The phototherapy device includes a number of projections positioned to press against and to illuminate a user's scalp, while reducing the presence of air between the projections and the user's scalp, thereby increasing optical penetration to the user's scalp. By utilizing phototherapy device according to the present disclosure, the projections advantageously bypass the user's hair and pushing against (also referred to as compressing) the scalp to increase light penetration by mechanical optical skin clearing.

Furthermore, when projections are exposed to the open air before reaching the scalp (i.e., when the projections are not in contact with the surface of a 3D skin surface), light delivery is reduced due to the index of refraction mismatch between the projections and air, and between the air and skin surface. By utilizing projections that come into direct contact with the 3D skin surface, the index of refraction mismatch is reduced by light transitioning directly between the projections and the skin surface.

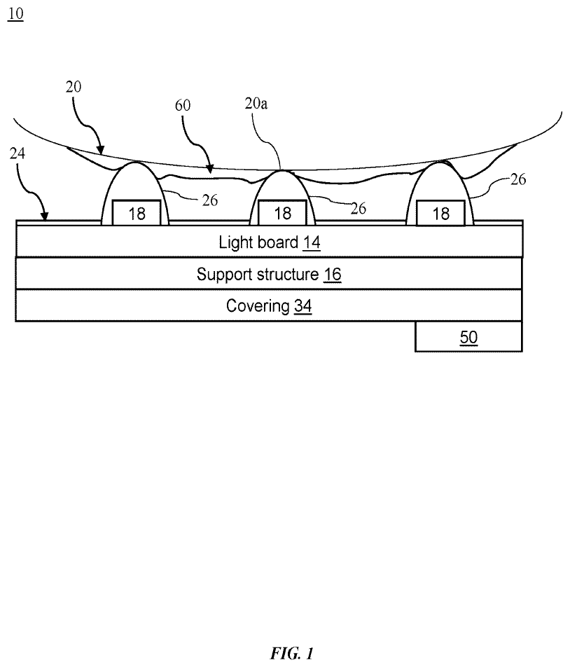

Turning to FIGS. 1-3, a phototherapy device 10 is shown for illuminating a three-dimensional (3D) skin surface 20. The phototherapy device 10 comprises an ellipsoidal dome 12 that includes a light board 14 mechanically supported by the supporting structure 16 and includes a plurality of light emitters 18 configured to emit light. The emitted light has a wavelength known to have a therapeutic effect on the 3D skin surface (such as stimulating hair growth).

The phototherapy device 10 further includes processor circuitry 22 configured to provide electrical power to each of the plurality of light emitters 18. As shown in FIG. 1, the phototherapy device 10 further includes an optically transparent elastomeric inner liner 24 including projections 26 applied to the light board 14. The light board 14 is positioned between the supporting structure 16 and the inner liner 24 with the light emitters 18 aligning with the projections 26.

The projections 26 are configured to improve light delivery to the 3D skin surface 20 by applying pressure to the 3D skin surface 20 when the ellipsoidal dome 12 is fit over the 3D skin surface 20, such that the projections 26 are pressed against the 3D skin surface 20 to improve an index of refraction matching between the projections 26 and the 3D skin surface 20 by reducing a presence of air between the projections 26 and the 3D skin surface 20. The ellipsoidal dome 12 may be shaped such that the projections 26 compress the 3D skin surface 20 to reduce an amount of body fluid in the 3D skin surface 20 at contact points 20a between the 3D skin surface 20 and the projections 26.

The use of compressive optical clearing according to the present disclosure may yield a decrease in the reflectance spectra of human skin in the spectral range 400-2000 nm. Analysis of this spectra based on the diffusion approximation of the radiation transfer theory has shown that the application of the external compression weakens the absorbing and scattering properties of user's skin tissue due to changes in the physiological properties of human skin in vivo.

The projections 26 may be made of any suitable material. For example, the projections 26 may be made of a polymer having an index of refraction more closely matching the index of refraction of skin than air. The projections 26 may also take any suitable shape. For example, the projections 26 may be generally cylindrical, conical, ellipsoidal, etc.

The phototherapy device 10 may be used in combination with an optical gel 60 for improving light delivery from the phototherapy device 10 to the 3D skin surface 20. The optical gel 60 may be configured to be applied to the 3D skin surface 20, such that delivery of the light emitted by the plurality of light emitters is improved due to improved index matching between the projections 26 and the 3D skin surface 20 by locating the optical gel 60 between the projections 26 and the 3D skin surface 20. The optical gel being located between the projections 26 and the 3D skin surface 20 reduces a presence of air between the projections 26 and the 3D skin surface 20, which improves light penetration into the skin surface 20.

The optical gel 60 may be a topical therapeutic including therapeutically active ingredients configured to promote a therapeutic effect (i.e., hair growth) on the 3D skin surface 20. The topical therapeutic may also include an optically clear excipient. The optical gel 60 may comprise any suitable gel, liquid, or foam having an index of refraction closer to the projections and/or the 3D skin surface than air.

The phototherapy device 10 may be used for any suitable therapeutic application. For example, the phototherapy device 10 may be used to treat psoriasis, hair loss, and other issues of the scalp. Additionally, the phototherapy device 10 may be used for transcranial photobiomodulation, e.g., to treat stroke, chronic traumatic brain injury, major depression, dementia, Parkinson's disease, bipolar disorder, anxiety, post-traumatic-stress disorder, consciousness disorders, autism, down syndrome, multiple sclerosis, amyotrophic lateral sclerosis, etc.

The topical therapeutics may include any formulation aimed at promoting hair regrowth. For example, the topical therapeutic may include at least one of minoxidil, finasteride, ketoconazole, saw palmetto extract, pumpkin seed oil, and biotin. These components can be singularly used or combined in various concentrations to tailor the formulation to specific hair regrowth needs.

The ellipsoidal dome 12 may be configured to fit snugly over the 3D skin surface 20 to provide occlusion of the topical therapeutic to enhance absorption or penetration of the therapeutically active ingredients by the 3D skin surface 20. The ellipsoidal dome 12 may also be configured to retain a portion of thermal energy generated by the light emitters 18, such that the retained thermal energy is applied to the 3D skin surface 20 to enhance the absorption or the penetration of the therapeutically active ingredients by the 3D skin surface 20.

For example, the ellipsoidal dome 12 of the phototherapy device 10 may include a covering 34 configured to act as a barrier between an external environment and an internal environment bounded by the 3D skin surface 20. The covering 34 may be configured to retain a portion of thermal energy generated by the light board 14 (e.g., the light emitters) such that the retained thermal energy is more effectively applied to the 3D skin surface 20. The covering 34 may be configured to improve absorption of topical therapeutics applied to the 3D skin surface 20 by providing warmth and occlusion.

The processor circuitry 22 of the phototherapy device 10 may be configured to modulate a thermal energy output by the plurality of light emitters 18 by intermittently providing electrical power to the plurality of light emitters 18 at a duty cycle. For example, the processor circuitry 22 may alter the duty cycle, such that during an initial time period an initial duty cycle is used to increase a temperature of the 3D skin surface 20. Following this initial time period, a subsequent duty cycle may be used that is less than the initial duty cycle. In this way, the higher initial duty cycle may be used to raise a temperature of the 3D skin surface 20 and the lower subsequent duty cycle may be used to maintain the raised temperature of the 3D skin surface.

In one embodiment, the phototherapy device 10 includes a temperature sensor configured to provide a temperature reading to the processor circuitry 22. The processor circuitry 22 may use the temperature sensor output to modulate the electrical power supplied to the light emitters 18 (e.g., such that the measured temperature is maintained near a preset value).

The processor circuitry 22 may have various implementations. For example, the processor circuitry 22 may include any suitable device, such as a processor (e.g., CPU), programmable circuit, integrated circuit, memory and I/O circuits, an application specific integrated circuit, microcontroller, complex programmable logic device, other programmable circuits, or the like. The processor circuitry 22 may also include a non-transitory computer readable medium, such as random access memory (RAM), a read-only memory (ROM), an erasable programmable read-only memory (EPROM or Flash memory), or any other suitable medium. Instructions for performing the method described below may be stored in the non-transitory computer readable medium and executed by the processor circuitry 22. The processor circuitry 22 may be communicatively coupled to the computer readable medium and network interface through a system bus, mother board, or using any other suitable structure known in the art.

In one embodiment, by overdriving the light emitters 18 (e.g., increasing peak power), photon penetration depth through the 3D skin surface 20 can be increased while simultaneously reducing the duty cycle. Accordingly, consistent overall power may be maintained, allowing for easier management of the phototherapy device 10 and its corresponding duty cycle and photon penetration. For example, while overdriving the light emitters for high peak power and reducing duty cycle (e.g., by overdriving a 5 mw laser to 10 mw and reducing the duty cycle from 100% to 50%) in combination with the techniques described in the present disclosure, overall photon penetration may be increased.

The supporting structure 16 of the phototherapy device 10 may be adjustable to illuminate 3D skin surfaces 20 having different shapes and sizes. For example, the supporting structure 16 may comprise an elastomeric cap 30 configured to stretch to fit over the 3D skin surface 20 to generate a force pressing the projections 26 against the 3D skin surface 20. Alternatively, the supporting structure 16 may be comprised of any elastomeric material to allow the phototherapy device 10 to conform to various head shapes and sizes.

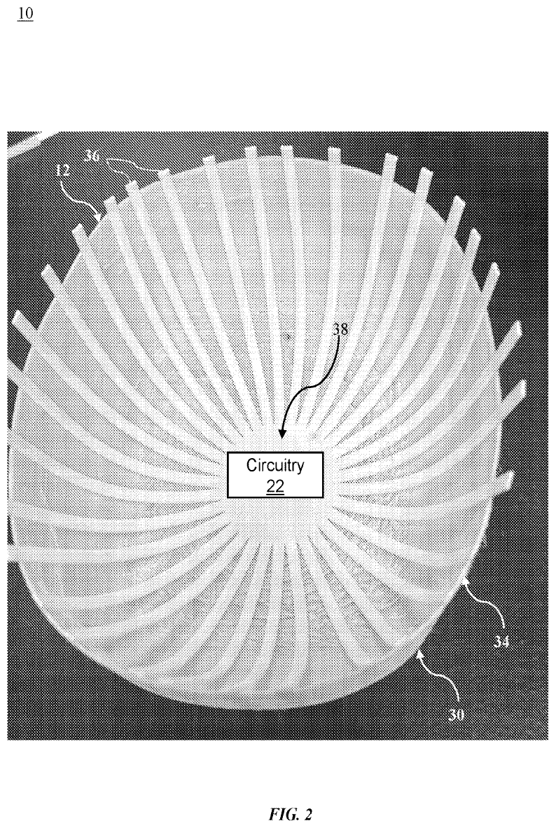

The supporting structure 16 of the phototherapy device 10 may include any suitable configuration for supporting the light emitters 18. For example, as shown in FIG. 2, the supporting structure 16 may include a flexible or winged light board 14 or structural elements 36 (e.g., spokes) radiating from a hub or central point 38. The light emitters 18 may be supported by and spaced along the structural elements 36 (i.e., spokes).

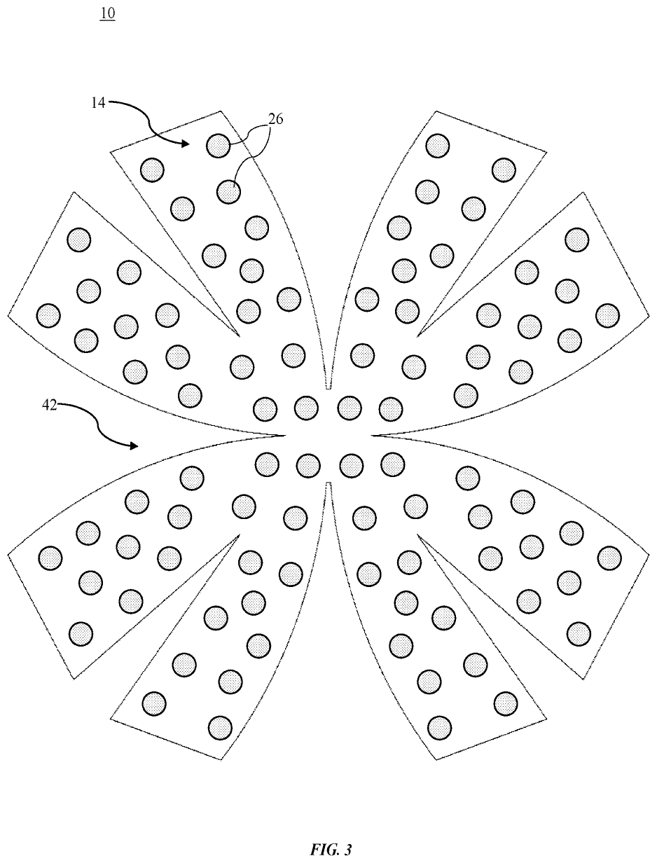

As shown in FIG. 3, the supporting structure 16 may alternatively incorporate flat manufactured lightboard configurations that further include cuts 42 spaced throughout with the lightboard segments 14 removed in the 2D lightboard structure running from the hub or central point 38 to enable the supporting structure 16 to form a half dome shaped elastomeric enclosure to allow the supporting structure 18 to stretch at certain points at the base of supporting structure 16.

The spacing between at least some of the structural elements may be adjustable to accommodate to provide illumination of varying and/or more complex 3D skin surface 20 shapes and sizes. By providing spaced cuts 42 in the lightboard 14 running outward from the central point 38 the flexible light board 14 may advantageously adjust in size and/or stretch with the elastomeric cap 30 to accommodate to provide illumination of varying and/or more complex 3D skin surface 20 shapes and sizes.

The light emitters 18 may be any suitable source of light (e.g., laser diodes, light emitting diodes (LEDs), etc.) that may emit electromagnetic radiation within a range of wavelengths or set of wavelengths having a known therapeutic effect. The light emitters 18 may emit electromagnetic radiation having wavelengths chosen from at least one of the set of ultraviolet, visible, and infrared.

In one embodiment, the supporting structure 16 includes an inflatable structure configured to receive air. As the inflatable structure receives air and increases in volume, the inflatable structure generates a force pressing the projections 26 against the 3D skin surface 20. By utilizing compressive optical clearing (i.e., by pressing the projections 26 against the 3D skin surface 20, photon penetration may be increased (e.g., by 10, 100, or 1,000 times).

The phototherapy device 10 may include a power supply 50 configured to supply electrical power to the processor circuitry 22. The power supply 50 may be any suitable source of electrical energy (e.g., battery, wall plug, etc.).

All ranges and ratio limits disclosed in the specification and claims may be combined in any manner. Unless specifically stated otherwise, references to “a,” “an,” and/or “the” may include one or more than one, and that reference to an item in the singular may also include the item in the plural.

Although the invention has been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A phototherapy device configured to illuminate a three-dimensional (3D) skin surface, comprising:
an ellipsoidal dome including:
a supporting structure;
a light board mechanically supported by the supporting structure and including a plurality of light emitters configured to emit light;
processor circuitry configured to provide electrical power to each of the plurality of light emitters; and
an optically transparent elastomeric inner liner including projections applied to the light board, such that the light board is positioned between the supporting structure and the inner liner with the light emitters aligning with the projections;
wherein a combined light emitted by the light emitters illuminates the surface of the 3D skin surface when positioned adjacent the 3D skin surface; and
wherein the projections are configured to improve light delivery to the 3D skin surface by applying pressure to the 3D skin surface when the ellipsoidal dome is fit over the 3D skin surface, such that the projections are pressed against the 3D skin surface improving an index of refraction matching between the projections and the 3D skin surface by reducing a presence of air between the projections and the 3D skin surface.

2. The phototherapy device of claim 1, wherein the ellipsoidal dome is shaped, such that the projections compress the 3D skin surface to reduce an amount of body fluid in the 3D skin surface at contact points between the 3D skin surface and the projections.

3. The phototherapy device of claim 1, wherein each of the projections is:
positioned to receive light emitted by a corresponding one of the light emitters; and
configured to act as a light guide to direct light emitted by the corresponding light emitter away from the light emitters and towards the 3D surface.

4. The phototherapy device of claim 1, wherein the supporting structure comprises an elastomeric cap configured to stretch to fit over the 3D skin surface to generate a force pressing the projections against the 3D skin surface.

5. The phototherapy device of claim 1, wherein the supporting structure comprises an inflatable structure configured to receive air and generate a force pressing the projections against the 3D skin surface upon the supporting structure being inflated.

6. The phototherapy device of claim 1, wherein the processor circuitry is further configured to modulate a thermal energy output by the plurality of light emitters by intermittently providing the electrical power to the plurality of light emitters at a duty cycle, such that:
during an initial time period an initial duty cycle is used to increase a temperature of the 3D skin surface; and
following the initial time period a subsequent duty cycle is used that is less than the initial duty cycle.

7. The phototherapy device of claim 1, wherein the ellipsoidal dome includes a covering configured to:
act as a barrier between an external environment and an internal environment bounded by the 3D skin surface and the covering;
retain a portion of thermal energy generated by the light board such that the retained thermal energy is applied to the 3D skin surface.

8. The phototherapy device of claim 7, wherein the covering is configured to improve absorption of topical therapeutics applied to the 3D skin surface by providing warmth and occlusion.

9. The phototherapy device of claim 1, wherein the support structure is adjustable to illuminate the surface of 3D skin surfaces having different sizes.

10. The phototherapy device of claim 1, further comprising a power supply configured to supply electrical power to the processor circuitry.

11. The phototherapy device of claim 1, wherein the plurality of light sources emit electromagnetic radiation within a range of wavelengths or set of wavelengths having a known therapeutic effect.

12. The phototherapy device of claim 11, wherein the plurality of light sources emit electromagnetic radiation having wavelengths chosen from at least one of the set of ultraviolet, visible, and infrared.

13. The phototherapy device of claim 1, wherein:
the supporting structure includes a hub and spokes radiating from the hub; and
the plurality of light emitters are supported by and spaced along the spokes.

14. The phototherapy device of claim 1, wherein the ellipsoidal dome has a reflective internal surface.

15. A phototherapy system for illuminating a three-dimensional (3D) skin surface, comprising:
an ellipsoidal dome including:
a supporting structure;
a light board mechanically supported by the supporting structure and including a plurality of light emitters configured to emit light;
processor circuitry configured to provide electrical power to each of the plurality of light emitters; and
wherein a combined light emitted by the light emitters illuminates the surface of the 3D skin surface when positioned adjacent the 3D skin surface; and
an optical gel for improving light delivery from the phototherapy device to the 3D skin surface, wherein the optical gel is configured to be applied to the 3D skin surface, such that light scattering by the 3D skin surface is reduced and transparency of the 3D skin surface is increased, resulting in improved delivery of the light emitted by the plurality of light emitters.

16. The system of claim 15, wherein the optical gel comprises a topical therapeutic including therapeutically active ingredients configured to promote a therapeutic effect on the 3D skin surface.

17. The system of claim 16, wherein the therapeutic effect includes hair growth.

18. The system of claim 13, wherein the ellipsoidal dome is configured to fit snugly over the 3D skin surface to provide occlusion of the topical therapeutic to enhance absorption or penetration of the therapeutically active ingredients by the 3D skin surface.

19. The system of claim 15, wherein the ellipsoidal open dome is further configured to retain a portion of thermal energy generated by the plurality of light emitters, such that the retained thermal energy is applied to the 3D skin surface to enhance the absorption or the penetration of the therapeutically active ingredients by the 3D skin surface.

20. The system of claim 16, wherein the topical therapeutic includes an optically clear excipient.
